(19) United States
(12) Patent Application Publication
(10) Pub. No.: US 2011/0030681 A1
(43) Pub. Date: Feb. 10, 2011

(54) DRUG DELIVERY DEVICE

(75) Inventors: Karin DE VRIES, Utrecht (NL); Wim JANSEN, LIESHOUT (US)

Correspondence Address: HOWREY LLP-EU, C/O IP DOCKETING DEPARTMENT, 1299 Pennsylvania Avenue, NW, Room B-3, Washington, DC, 20004-2402, US

(73) Assignee: N.V. ORGANON, OSS (NL)

(21) Appl. No.: 12/297,689

(22) PCT Filed: Apr. 17, 2007
(86) PCT No.: PCT/EP07/53734

§ 371 Date: Oct. 20, 2008

Related U.S. Application Data
(63) Continuation of application No. PCT/EP2007/053734, filed on Apr. 17, 2007.

(30) Foreign Application Priority Data
Apr. 19, 2006 (EP) 06 112 477.5
Oct. 13, 2006 (EP) 06 122 293.1

(51) Int. Cl. A61M 15/00

(52) U.S. Cl. 128/203.12

(57) ABSTRACT

A dispenser for administering a pharmaceutical substance in the form of a plurality of separate unit doses of the substance, comprising a housing, an actuator, a delivery outlet for administering at least one unit dose at a time, a transport mechanism for bringing the unit doses sequentially in operative association with the delivery outlet and a delaying arrangement to impose a lockout interval between administering sequential doses

### CROSS REFERENCE TO RELATED APPLICATIONS

This application is a continuation of International Application No. PCT/EP2007/053734 which was published under PCT Article 21(2) in English, the contents of which are hereby incorporated by reference in their entirety.

### BACKGROUND OF THE INVENTION

1. Field of the Invention

The present invention relates to a dispenser for administering a pharmaceutical substance, in particular in the form of a liquid, having a delaying arrangement to impose a lockout interval, i.e., a predetermined interval of time, between administering sequential doses. The invention further relates to a strip of unit doses for use in the dispenser.

2. Description of the Related Art

Dispensers of the aforementioned kind allow for self-administering a pharmaceutical substance, such as an analgesic or an insomnia medicine. A lockout interval assists the prevention of overdosing of and addiction to the substance, it also renders the dispenser more child-safe.

In the art, such dispensers are particularly adapted for use with infusion or intravenous medication systems (IV systems). The conflicting aspects of user-friendliness and reliability of the dispenser, accuracy of the doses to be dispensed as well as reliability of the lockout interval result in technically complex devices and/or devices which do not fully inhibit dispensing of additional substance within the intended lockout interval.

For instance, U.S. Pat. No. 4,828,551 describes a patient controlled analgesia (PCA) apparatus, which is mechanical/hydraulic and comprises a reservoir and a pump operable by the patient for dispensing medicine from the reservoir unto the patient's IV system in incremental doses. The pump capacity per stroke may be adjustable, thereby adjusting the size of each dose dispensed. A timing apparatus assures that a specific interval of time (the lockout interval) must pass between sequential dosage dispensations.

WO 2005/089836 describes an apparatus and a method, which are provided for dispensing a liquid, such as an analgesic that has to be administered to a patient. The apparatus includes a reservoir filled with an analgesic that is connected via a handset, to administering means such as a needle or cannula. The handset includes inlet and discharge valves as well as a resilient, porous recoil structure. The analgesic flows from the reservoir through the inlet valve into the pores of the recoil structure and flow from the recoil structure through the discharge valve to be administered to the patient. The patient can compress the recoil structure to administer a bolus dose of the analgesic and when the recoil structure is released, its recoil nature draws the liquid from the reservoir into and filling its pores. This flow is limited not to exceed a predetermined maximum rate.

U.S. Pat. No. 6,010,483 describes a pump or method for supplying patient controlled analgesia to a patient. The infusion flow rate of the analgesic is increased in response to a command given by the patient receiving the analgesic, and a lockout interval is provided during which time the actuation of the pump by the patient produces no further incremental increase in the flow rate of the analgesic.

For some pharmaceutical substances, e.g. for treating insomnia, diabetes and/or psychosis, administration by means of an infusion system is unnecessary or undesired and an oral spray may be a preferred administration form. An oral spray is also easier to swallow than pills, shows a rapid bioavailability and thus can result in a more instant effect of the composition than a medicine in pill or capsule form.

Existing spray bottles, however, suffer from a lack of accuracy in the dispensed dose, as the suction tube and/or the reservoir can be filled with air so that no full dose is dispensed in one actuation stroke. Also, a partial or insufficiently forceful operation of the actuator results in a too low dose. This may cause the user to attempt more than one dispense, yielding a too high dose.

### BRIEF SUMMARY OF THE INVENTION

It is therefore an object of the present invention to provide an improved dispenser.

To this end, the dispenser comprises a plurality of separate unit doses of the substance, and a delivery means, preferably a spray nozzle, for administering at least one, preferably precisely one unit dose at a time.

It is preferred that the delaying means comprises a mechanical timing system, preferably comprising a main spring, a gear train and an escapement. Thus, a reliable and relatively inexpensive lockout timer is provided which is operable by a user and which does not require an additional power supply such as a battery, which may discharge, e.g. during prolonged storage thus effectively reducing the shelf-life of the dispenser. A mechanical timing system is also insensitive to electromagnetic signals and/or noise.

It is preferred that the unit doses are interconnected by and/or are part of a strip. A strip provides a convenient package for a plurality of unit doses, for instance intended for or representing a full course of treatment or a particular period of time, i.e. one month.

It is preferred that the dispenser comprises a transport mechanism for bringing the unit doses sequentially in operative association with the delivery means and, preferably, for bringing the strip stepwise from a first spool to a further spool. In this way it may be ensured that a new unit dose is available for each administration. A spool provides a convenient and relatively low-volume means for the storage of a strip wound thereon. Providing two spools allows to easily contain both the full and emptied parts of the strip inside the dispenser.

In a preferred embodiment, the mechanical timing system is coupled to the transport mechanism such that the main spring of the mechanical timing system is wound by and/or during transport of the strip and the lockout interval is initiated by operating the actuator. Thus, the timing system of the dispenser is “armed” during the transportation of a fresh unit dose to the delivery means, in preparation for the administration of a dose. Upon administration of a dose by the operation of the actuator the timing of the lockout period by the timer is started, so that dispensing is prohibited as no new doses can become available. After the lockout interval has elapsed the dispenser is again ready for the administration of another dose, starting the cycle anew.

It is preferred that during the lockout interval, the mechanical timing system locks the transport mechanism.

This prevents to prepare a new dose for dispensing prior to the end of the lockout interval, thus preventing accidental premature release of a dose and possible misuse.

The invention will now be explained in more detail and with reference to the Figures, which schematically show a presently preferred embodiment of the dispenser according to the invention comprising a mechanical timing system.

### BRIEF DESCRIPTION OF THE DRAWINGS

FIG. 1 is a perspective view of a dispenser according to the present invention.

FIG. 2 is a perspective view of the dispenser of FIG. 1, of which a front part of the housing is removed.

FIG. 3A shows a side view of the front half-shell of the dispenser of FIGS. 1 and 2.

FIG. 3B shows a cross-sectional view of the front half-shell of the dispenser of FIG. 3A along the line IIIB.

FIG. 3C shows an enlarged part of the cross-section of FIG. 3B.

FIG. 4 shows an exploded view of the transport mechanism and the lockout system.

FIG. 5 shows the timing system of the dispenser.

FIG. 6 shows a flow chart of the operation of the dispenser.

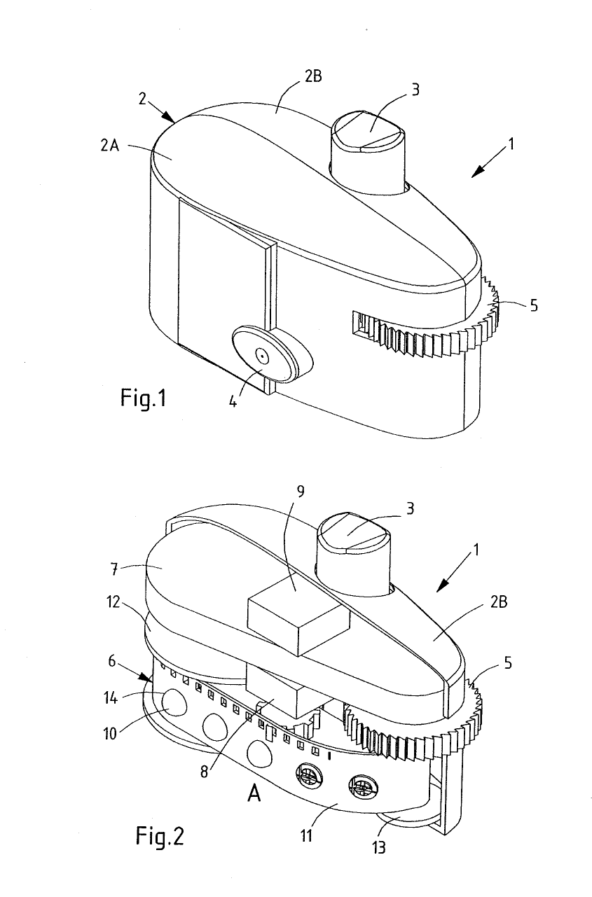

FIG. 1 shows a dispenser 1 comprising a housing 2, here comprising two half-shells 2A and 2B, respectively. The dispenser further comprises an actuator in the form of a push button 3, a spray nozzle 4 and a dose transport wheel 5. As schematically shown in FIG. 2, inside the housing 2 of the dispenser 1 a dose transport mechanism 6, and a timing system or timer 7 are arranged, as well as a transport blocking mechanism 8 and a timer blocking mechanism 9.

The dose transport assembly 6 comprises a plurality of unit doses 10, which are part of a strip 11, and a first and second spool 12, 13, on which the strip 11 is wound. The letter A indicates the position of the spray nozzle 4.

The strip 11 is a flexible strip comprising a film or laminate in- or onto which blisters 14 have been formed in each of which a unit dose 10 is contained.

The part of the strip 11 containing the unit doses 10 is wound on the first spool 12, the second spool 13 is provided for collecting the empty strip 11.

The dose transport assembly 6 provides a mechanism for bringing the unit doses 10 sequentially in operative association with the spray nozzle 4 and for bringing the strip 11 stepwise from the first spool 12 to the further spool 13.

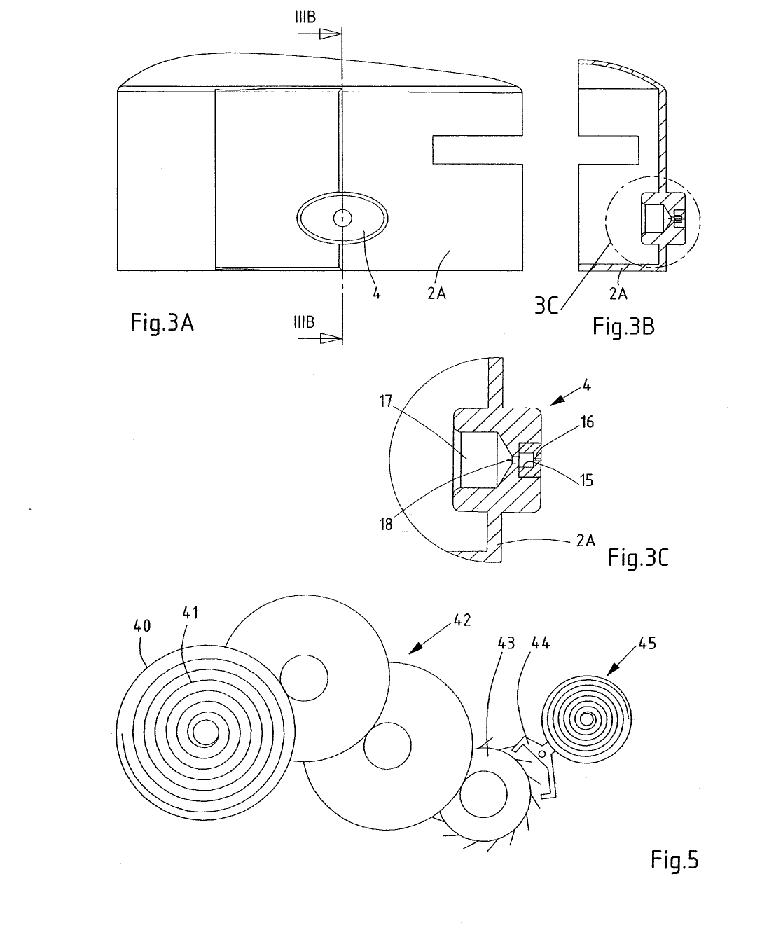

The spray nozzle 4 is arranged or formed in the front wall of the housing 2 and comprises a swirl chamber 15 and a nozzle channel 16, as shown in detail in FIG. 3C. Part of the nozzle 4 extends into the housing 2 of the dispenser and defines a dose cavity 17. A sharp protrusion 18 is located adjacent the channel between the dose cavity 17 and the swirl chamber 15.

For the administration of a dose, e.g. as an oral spray, a unit dose 10, here comprised in a blister 14 is brought in register with the nozzle 4, more specifically in this embodiment in register with the dose cavity 17. By operating the push button 3, a plunger (not shown) is actuated inside the dispenser 1 for pressing the blister 14 containing the dose 10 into the dose cavity 17. Due to the pressure exerted on the blister 14 by the plunger and, in this embodiment, assisted by the sharp protrusion 18, the blister 14 ruptures and releases the liquid of the dose 10 into the swirl chamber 15, yielding a fine spray from the nozzle channel 16.

The volume of the unit dose is typically in a range from 0.02 ml to 0.5 ml, preferably in a range from 0.05 ml to 0.2 ml.

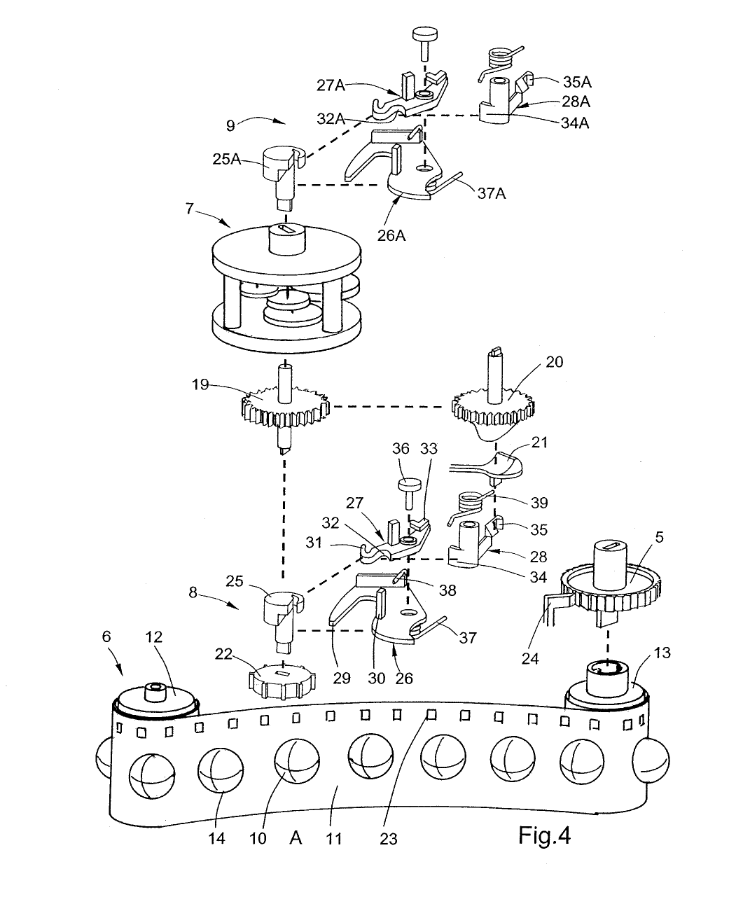

FIG. 4 shows an exploded view of an embodiment of the dose transport mechanism 6, the timing system 7, the transport blocking mechanism 8 and the timer blocking mechanism 9, as well as a first and a second gear 19, 20, a release lever 21 and a toothed wheel 22, which interconnect the abovementioned parts. The toothed wheel 22 grips into slots 23 which are provided along at least one of the edges of the strip 11.

The transport and the timer blocking mechanisms 8, 9 of this embodiment are substantially identical. Equivalent elements are indicated with the same reference numeral, with the elements of the timer blocking mechanism 9 carrying an additional suffix “A”. The operation of the blocking mechanisms 8, 9 will be explained hereafter with reference to the dose transportation.

The dose transportation assembly comprises the dose transport wheel 5, the dose transport mechanism 6, the transport blocking mechanism 8 and the toothed wheel 22. The assembly further comprises a flag 24, which is resiliently mounted in contact with the saw-toothed rim of the dose transport wheel 5.

The transport blocking mechanism 8 is similar to that of a photo-camera and comprises a locking wheel 25, a lever 26, a gripper 27, and a hammer 28. The head of the locking wheel 25 is provided with a recess and an asymmetric side, here at the bottom. The lever 26 comprises an arm 29 and a post 30. The gripper 27 comprises a hook 31, a recess 32 and a rear arm 33. The hammer 28 comprises a flag 34 and an arm 35. The lever 26 and the gripper 27 are pivotably mounted on one pin 36. The lever 26 is spring tensioned around the pin 36 in the direction of the arm 29 with a spring 37. The gripper 27 is spring tensioned around pin 36 in the direction of the hook 31 with the spring arm 38 acting on the rear arm 33. The spring arm 38 is the upper end of the lever spring 37. The hammer 28 is spring tensioned around an axis (not shown) with a spring 39 in the direction of the flag 34.

In the initial, unblocked, situation the lever 26 is arranged so that the arm 29 extends underneath the head of the locking wheel 25, the gripper 27 is urged against the post 30 by the spring arm 38, the hook 31 is free from contact with the locking wheel 25, the flag 34 of the hammer 28 protrudes into the recess 32 of the gripper 27 and the arm 35 of the hammer 28 is free from the dose transport wheel 5.

To transport a new unit dose 10 to the position A of the nozzle 4, the dose transport wheel 5 is turned so that the strip 11 is wound up on the second spool 13 and unwound from the first spool 12. The transportation direction is determined by the flag 24 slipping past or engaging the saw-toothed rim of the dose transport wheel 5.

The transport motion of (the slots 23 of) the strip 11 causes a rotation of the toothed wheel 22 and of the connected locking wheel 25. The lower part of the head of the blocking wheel 25 engages the arm 29 of the lever 26 so that the lever 26 pivots so far about pin 36 that the gripper 27 no longer rests against the post 30 but that it abuts the rim of the locking wheel 25 with the hook 31.

The distance between adjacent doses 10 along the strip 11 corresponds to a full revolution of the toothed wheel 22 and of the connected locking wheel 25, allowing the hook 31 to engage the recess in the locking wheel 25. This causes the flag 34 of the hammer 28 to slip past the edge of the recess 32 and to press against the side of the gripper 27, which in this position again abuts the post 30. Furthermore, the tip of the arm 35 of the hammer lodges in the teeth of the dose transport wheel 5. In this position further rotation of the locking wheel 25 and the connected toothed wheel 22, as well as of the dose transport wheel 5 is inhibited, thus halting further transport of the strip 11.

The transport blocking is removed by applying a vertical pressure against the wedge-shaped portion of the arm 35 of the hammer 28, pivoting the hammer 28 about its axis (not shown). The rotation of the hammer 28 removes the arm 35 from the teeth of the dose transport wheel 5 and causes the flag 34 to slip into the recess 32 of the gripper 27, enabling the lever 26 and the gripper 27 to pivot back to their initial, unblocking, position, due to the spring 37.

The timing system 7 is coupled to the transport mechanism by means of the locking wheel 25 and the toothed wheel 22. An embodiment of the timing mechanism is shown in FIG. 5 and comprises a power wheel 40 with a main spring 41, a gear train 42 comprising several gears, an escapement wheel 43, an anchor 44 and a balance wheel with a balance spring 45.

The main spring 41 is wound by transport of the strip by means of the revolution of the toothed wheel 22, which causes a revolution of the power wheel 40. The locking wheel 25A is connected to the power wheel 40 and is rotated simultaneously. After a full revolution of the power wheel 40 the timing mechanism 7 is blocked by the timer blocking mechanism 9, by engaging the locking wheel 25A with the gripper 27A and placing a bar (not shown) attached to the arm 35A of the hammer 28A between the teeth of the escapement wheel 43.

The lockout interval is initiated by operating the actuator 3. Applying a vertical pressure with the actuator 3 against the wedge-shaped portion of the arm 35A of the hammer 28A causes the hammer 28A to pivot about its axis (not shown). The rotation of the hammer 28A removes the bar from the teeth of the escapement wheel 43 and causes the flag 34A to slip into the recess 32A of the gripper 27A, enabling the lever 26A and the gripper 27A to pivot back to the initial, unblocking, position due to the spring 37A.

The lockout interval is the duration of a full revolution of the power wheel 40, determined by the unwinding of the main spring 41 and the concerted motion of the other elements of the timing mechanism 7. The lockout interval may be set with the relative sizes of the cogs of the gear train 42. Preferably, but depending on the employed pharmaceutical substance, the lockout interval lasts at least one, preferably at least two hours. Another preferred lockout interval is in a range from 4 to 24, more preferably from 4 to 18 hours, e.g. a lockout interval of 6 hours for a preparation that should be taken three times a day, i.e. every 8 hours, or a lockout interval of 10 hours for a preparation that should be taken two times a day, i.e. every 12 hours. A lockout interval of 22 hours is especially suitable for an insomnia drug or for a preparation that should be taken once a day, e.g. an oral contraceptive.

The full revolution of the power wheel 40 during the lockout interval causes, via the first gear wheel 19, a full revolution of the second gear wheel 20. The second gear wheel 20 has a revolving slope at the bottom, which abuts the sloped side of the release lever 21, pressing it down at the end of the revolution. A pin underneath the release lever 21 then presses against the wedge shaped portion of the arm 35 of the hammer 28 and causes the hammer 28 to pivot, thus unblocking the transport blocking mechanism 8, as described above.

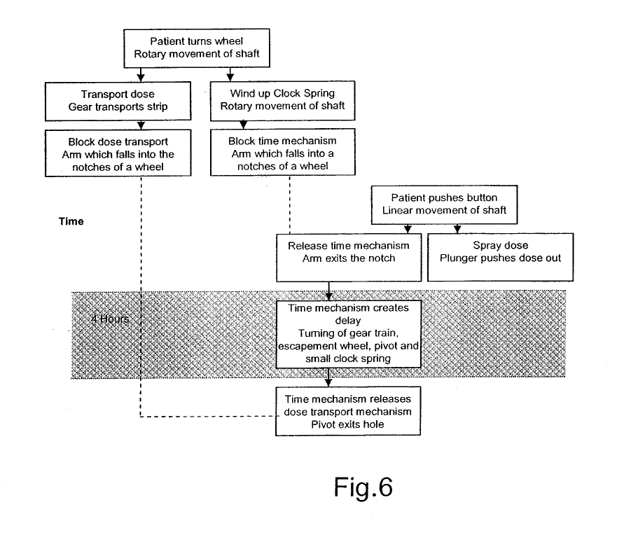

In sum, the operation of the dispenser 1 by a user comprises two main actions, as indicated in the flow chart of FIG. 6. First, the user turns the dose transport wheel 5, placing a new dose 10 in front of the nozzle 4 and at the same time winding up the (main spring 41 of the) mechanical timing mechanism 7. After a sufficient turn both blocking systems 8 and 9 are engaged. Second, the user presses the actuator 3, dispensing a dose 10 and at the same time resetting the timer blocking mechanism 9, thus unblocking the timing mechanism 7 and initiating the lockout interval. After the lockout interval has elapsed, the timing mechanism 7 resets the transport blocking mechanism 8, thus unblocking the transport mechanism 6 and resetting the dispenser 1 for the administration of a subsequent dose 10.

The inventive dispenser is portable and self-contained, i.e. it does not require further components to be able to perform its task or require connection to another system, such as an IV-system. However, the dispenser may be adapted for such systems.

The dispenser is very well suited for use with a pharmaceutical composition for treating a human or an animal. The dispenser is preferably used for a pharmaceutical composition comprising at least comprising an active ingredient for treating insomnia, diabetes and/or psychosis, for which a lockout interval between successive doses is desired. Similarly, the dispenser may be used for the administration of oral contraceptives.

The invention is not restricted to the above-described mechanical embodiment, but can be varied in a number of ways within the scope of the claims.

For instance, a different mechanical or electrical timing mechanism, transport mechanism and/or blocking mechanism may be employed, as well as mutually different blocking mechanisms.

In a particular embodiment the dispenser, preferably included in and/or coupled with the dose transport system, is provided with a generator such as a dynamo for generating electrical energy for operating an electronic lockout timing mechanism. Such an embodiment is preferably provided with an indicator, such as an LED, for signalling correct operation and/or that sufficient energy has been generated.

Further, the dispenser can be adapted to dispense powders, pills or capsules from a delivery means such as a spout or a simple orifice in the housing. The dispenser may also be provided with a connection for an injection needle, to enable intravenous administration of the unit dose.

The unit doses may also be interconnected as, possibly detachable, links of a chain. Similarly, the unit doses contained in one strip or chain need not be equal; various treatments, compositions or concentrations corresponding to a particular treatment or regimen or a phase thereof may be administered with the same device, e.g. for the different phases of a cycle in the case of a contraceptive.

The dispenser can also be provided with an indicator for indicating the number of doses spent and/or remaining inside the device.

What is claimed is:

1. A dispenser for administering a pharmaceutical substance in the form of a plurality of separate unit doses of the substance, comprising:
a housing;
an actuator;
a delivery outlet for administering at least one unit dose at a time;
a transport mechanism for bringing the unit doses sequentially in operative association with the delivery outlet; and
a delaying arrangement to impose a lockout interval between administering sequential doses, wherein the delaying arrangement comprises a mechanical timing system coupled to the transport mechanism such that a main spring of the mechanical timing system is wound by or during transport of the doses and the lockout interval is initiated by operating the actuator.

2. Dispenser according to claim 1, wherein the lockout interval lasts at least one hour.

3. Dispenser according to claim 2, wherein the lockout interval is in a range from 4 to 18 hours.

4. Dispenser according to claim 1, wherein the mechanical timing system, comprises a gear train, and an escapement.

5. Dispenser according to claim 1 further comprising a strip of unit doses, wherein the unit doses are interconnected by or are part of the strip.

6. Dispenser according to claim 5, wherein the strip is flexible and comprises at least one film or laminate in- or onto which blisters have been formed and wherein unit doses are contained in the respective blisters.

7. Dispenser according to claim 6, wherein the strip containing the unit doses is wound on a spool.

8. Dispenser according to claim 7, comprising a further spool for collecting the empty strip.

9. Dispenser according to claim 8, wherein the transport mechanism is arranged for bringing the strip stepwise from the first spool to the further spool.

10. Dispenser according to claim 1, wherein, during the lockout interval, the mechanical timing system locks the transport mechanism.

11. Dispenser according to claim 1, wherein the dispenser is portable and self contained.

12. Dispenser according to claim 1 further comprising a strip of unit doses, wherein the volume of the unit dose is in a range from 0.02 ml to 0.5 ml.

13. Dispenser according to claim 12, which unit dose contains a pharmaceutical composition at least comprising an active ingredient for treating insomnia, diabetes or psychosis.

14. Dispenser according to claim 12, which unit dose contains an oral contraceptive.

15. Dispenser according to claim 1 further comprising a strip of unit doses of the substance in liquid form.

16. Dispenser according to claim 1, wherein the actuator is manually actuated.

17. Dispenser according to claim 1, wherein the delivery outlet is a spray nozzle.

18. A strip for use in the dispenser as claimed in claim 1, comprising a plurality of separate unit doses of a liquid pharmaceutical substance comprised in a blister, the blister being arranged to rupture on application of pressure to release the liquid for delivery, wherein the strip comprises a plurality of slots provided along at least one of its edges for engaging with the transport mechanism.

19. The strip according to claim 18, wherein a distance between adjacent doses on the strip corresponds to a full revolution of a locking wheel of the mechanical timing system.

20. The strip according to claim 18, further comprising a spool on which the strip is wound.

21. A dispenser for administering a liquid pharmaceutical substance in the form of a plurality of separate unit doses of the substance contained within blisters on a strip, the dispenser comprising:
a housing;
a manually actuated actuator arranged to cause rupture of a blister on application of pressure to release the liquid for delivery;
a delivery nozzle for administering at least one unit dose at a time;
a transport mechanism for bringing the unit doses sequentially in operative association with the delivery nozzle and the actuator; and
a delaying arrangement to impose a lockout interval between administering sequential doses, wherein the delaying arrangement comprises a mechanical timing system coupled to the transport mechanism such the mechanical timing system is armed by transport of the doses.
